(19) United States
(12) Patent Application Publication
(10) Pub. No.: US 2005/0277634 A1
(43) Pub. Date: Dec. 15, 2005

(54) Liquid composition for veterinary medicine and process for the preparation and use thereof

(75) Inventors: Dietrich W. Janott, Ingelheim (DE); Stefan Henke, Kirchen (DE); Martin Folger, Ingelheim (DE); Peter Riechel, Ingelheim (DE); Bernhard Hassel, Ockenheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Vetmedica GmbH, Ingelheim (DE)

(21) Appl. No.: 11/131,594

(22) Filed: May 18, 2005

(30) Foreign Application Priority Data
May 19, 2004 (DE) 10 2004 025 324

(51) Int. Cl. A61K 31/5415

(52) U.S. Cl. 514/226.5

(57) ABSTRACT

A liquid composition comprising: meloxicam, penethamate hydroiodide, and at least one solvent.

### RELATED APPLICATIONS

This application claims priority to German Application No. 10 2004 025 324.2, filed May 19, 2004, which is hereby incorporated by reference in its entirety.

### FIELD OF THE INVENTION

The invention relates to a liquid preparation for veterinary medicine, a process for preparing it, and the use thereof.

### BACKGROUND OF THE INVENTION

As a result of the breeding in the last few decades, attempting to achieve greater ease of milking in cows, the downside of this breeding is now becoming apparent in the form of increasing problems with inflammation of the udder (mastitis). Mastitis in cattle is the commonest reason for premature slaughtering of dairy cows, apart from fertility problems, in areas of intensive milk production. Consequently, the importance of drugs used in this field is growing.

Thus, meloxicam is a non-steroidal inflammation-inhibiting active substance of the oxicam category and is also marketed as a solution containing meloxicam (sold under the trademark Metacam®). It is a known cyclooxygenase (COX-I/COX-II) inhibitor which has the following chemical structure:

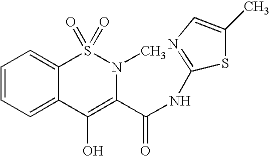

This non-steroidal anti-inflammatory is licensed inter alia for the treatment of mastitis in dairy cows, i.e., it is effective against inflammation of the udder. This drug is also used for acute cases, particularly for preventing long term inflammatory damage to the udder. In veterinary medicine meloxicam is used particularly as an injectable solution, the active substance being fully bioavailable after subcutaneous administration. Maximum plasma concentrations are achieved after a few hours. As a rule, a single injection is sufficient.

As all the main metabolites of meloxicam are pharmacologically inactive, the waiting time after an injection has been given is determined solely by means of the concentration of active substance in the tissue in question and, in the case of Metacam®, is 15 days for edible tissue in cattle and cows and 5 days for milk. In pigs a waiting time of 5 days is essential.

Other indications for this active substance in cattle and pigs are respiratory complaints in cattle and cows, diarrhea in calves, acute mastitis in dairy cows, MMA (mastitis-metritis-agalactica syndrome) in pigs, and non-infectious lameness in pigs.

Streptococi, Staphylococi, and E. coli have been diagnosed as the cause of acute mastitis. Therefore, treatment with meloxicam is frequently given in conjunction with an antibiotic, as a result of which the clinical symptoms improve even more significantly and there is rapid and lasting inhibition of the local inflammation of the udder tissue. In principle, β-lactam antibiotics, aminoglycosides, lincosamides, macrolides, cephalosporines, tetracyclines, penicillines, polypeptides, trimethoprim-sulfonamide combinations, and fluoquinolones are suitable for antibacterial mastitis therapy.

It is therefore useful to carry out a suitable antibiotic therapy at the same time as administering meloxicam. For this reason, the prior art contains numerous suggestions in a wide variety of medical fields for administering meloxicam in conjunction with antibiotics.

Thus, WO 01/015687 describes a combination of COX-II selective inhibitors such as meloxicam, optionally in conjunction with other active agents including antibiotics, for the treatment or prevention of prostatitis or chronic pain syndrome in the small pelvis.

WO 93/01814 describes an ophthalmological composition which can be instilled into the eye, containing an inflammation-inhibiting agent selected from among the oxicams together with a gel-forming acrylic acid polymer and a basic agent, while an antibacterial agent such as an antibiotic may also be present.

WO 98/06385 and EP 0 918 513 each relate to pharmaceutical compositions which are easy to swallow for oral administration containing at least one active substance, provided with corresponding coatings. Among a large number of possible active substances, meloxicam and antibiotics are also mentioned.

The development of special combined preparations is therefore always of major interest. In view of the importance of the health of farm animals for human consumption, there is always a need to find ways of treating diseases in these animals more effectively or preventing such diseases and providing suitable drugs for this purpose.

The objective of the present invention is therefore to provide a preparation by means of which meloxicam can easily be administered together with an antibiotic. The intention is to develop a formulation in which a combination of meloxicam and antibiotic is provided, where neither of the active substances is negatively affected in terms of its spectrum of activity. A further aim is to provide a process for preparing this formulation by simple means. In addition, the formulation to be developed should be easy to administer to animals.

### DETAILED DESCRIPTION OF THE INVENTION

The objective outlined above is achieved by means of the features of claim 1. According to this, a liquid preparation for veterinary medicine is provided, containing: as first active substance meloxicam; as second active substance penethamate hydroiodide; and at least one solvent.

Accordingly, a first active substance, i.e., meloxicam, is provided in conjunction with a particular antibiotic, i.e., penethamate hydroiodide, as second active substance, in the form of a liquid preparation. The second active substance, penethamate hydroiodide, also known as “benzylpenicillate hydroiodide” and “penethacillin hydroiodide”, is an active substance which is used in the treatment of mastitis in cattle triggered by streptococi and non-β-lactamase-forming staphylococci and other penicillin-sensitive pathogens. This active substance is marketed as an intramuscular mastitis treatment under the brand name Ingel-Mamyzin®, the active substance penethamate hydroiodide being supplied as a dry substance and the solvent in the form of water, containing methyl-4-hydroxybenzoate as preservative, being supplied separately, the two components only being mixed together immediately before use. The shelf life of the injectable solution of Ingel-Mamyzin® after the two components, i.e., the dry substance and solvent, have been mixed together is 7 days at a storage temperature of between 2° C. and 8° C. and 2 days at a storage temperature in the range from 15° C. to 25° C.

Therefore, the present invention has surprisingly succeeded, simply by replacing the solvent in the Ingel-Mamyzin® with a meloxicam solution, in producing a particularly effective liquid preparation. It has not hitherto been possible to treat mammals with a mixture of this kind as a meloxicam solution has a pH of around 9 and it has therefore been assumed that it would not be possible to obtain stable compositions. However, experiments which are described in detail in the Examples unexpectedly demonstrated that sufficient stability is achieved.

The liquid preparation according to the invention enables the two active substances to be administered simultaneously and develop their activities unimpeded.

The liquid preparation is obtained in the form of a suspension. It is a dispersed system. By a “dispersed system” is meant, in general terms, a systems consisting of two or more phases in which one type of form (dispersed phase) is finely divided in the other type of form (dispersion agent). By a “suspension” is meant, according to the invention, a mixture of solid particles in a liquid.

The suspension is preferably homogeneous, i.e., immediately after shaking the suspension appears to the observer to have a substantially uniform appearance. “Liquid preparation” does not, however, rule out the fact that solid particles may be precipitated and that this precipitate will increase as the suspension is left to stand and will disappear partly or entirely when shaken again. The term “liquid preparation” as used in the present invention simply means that the majority of the preparation is present in liquid form.

It has proved advantageous for the meloxicam in the liquid preparation to be dissolved in the solvent. “Dissolved” in this context not only refers to a genuine solution but also includes a dispersed system, particularly a colloidally dispersed system.

Expediently, the solvent used in the liquid preparation is selected from among alcohol, water and/or alcohol/water, of which ethanol is particularly preferred. Most particularly preferred are ethanol/water mixtures with an ethanol/water ratio (w/w) of from 1:7 to 1:5.7, preferably from 1:6 to 1:5.7, most preferably 1:5.7.

According to a preferred embodiment, the preparation contains 0.2 to 0.5, particularly 0.34 to 0.4 g of penethamate hydroiodide per mL of solution. Most preferably the penethamate hydroiodide is used as dry substance. “Dry substance” means that the penethamate hydroiodide is used in dried form, such as is already available as a component of Ingel-Mamyzin®.

It is also preferable to use a solution which corresponds to a dosage range of from 0.2 to 0.6 mg, preferably 0.5 mg, of meloxicam per kg of bodyweight and 8 to 12 mg, preferably 10 mg, of penethamate hydroiodide per kg of bodyweight.

It is also preferable if meloxicam is present in the liquid preparation according to the invention (final formulation) in an amount of from 0.2 g to 0.4 g per mL of final formulation, particularly 0.25 to 0.32 g per mL of final formulation.

Of course, excipients may also be present and these are not particularly restricted within the scope of the invention. Purely by way of example, the excipients in a standard commercial meloxicam solution for injection might be: meglumine, Macrogol 30D, Poloxamer 188, EDTA-Na, and glycine. Of course, other excipients known to the skilled man may also be used.

It is particularly advantageous if the liquid preparation is prepared in the form of an intramuscular injectable solution, optionally containing suitable excipients. This makes it possible to achieve accurate metering, easy administration and a rapid onset of activity.

A most particularly preferred liquid preparation of the invention contains
0.25 to 0.31 g of meloxicam per mL of final formulation;
0.40 to 0.30 g of penethamate hydroiodide per mL of final formulation;
1.88 to 0.34 mL of ethanol; and
7.74 to 0.67 mL of water.

The invention also relates to a process for preparing the liquid preparation described above, comprising the following steps:
(a) dissolving meloxicam in a solvent;
(b) adding penethamate hydroiodide to the solution obtained in step (a); and
(c) shaking the mixture obtained in step (b), to obtain the liquid preparation in the form of a suspension.

The process according to the invention thus provides a simple method of preparing a combined preparation, in which the combining of the two components followed by shaking of the mixture produces the preparation ready for use.

First of all, meloxicam is dissolved in a solvent, the term “dissolved” for the purposes of the invention also encompassing “dispersed”, as defined above. It is particularly preferable if the solution of meloxicam obtained in step (a) is prepared in a solvent such as ethanol and optionally water in a 1% to 10% concentration, more particularly 1% to 5%. Most preferably, a 2% meloxicam solution is used as the starting material. Of course, a standard commercial injectable solution of meloxicam may also be used.

Then, in step (b) according to the invention, the penethamate hydroiodide is added in the required amount, as explained previously, and is preferably used as a dry substance.

Naturally, in step (a) and/or step (b), the excipients described previously may be added in the amounts specified.

Advantageously, the liquid preparation according to the invention is prepared just before use. The shelf life after mixing is 7 days at a storage temperature of between 2° C. and 8° C.

The invention also relates to the use of the liquid preparation for producing a pharmaceutical composition for the simultaneous treatment or prevention of diseases in animals, selected from the known indications for meloxicam and/or penethamate hydroiodide, preferably selected from the indications of respiratory complaints in cattle and cows, diarrhea in calves, mastitis, acute mastitis in diary cows, mastitis-metritis-agalactica syndrome in pigs, non-infectious lameness in pigs, prostatitis, and chronic pain syndrome in the small pelvis.

One indication is the treatment of mastitis. However, it is also possible to treat two indications simultaneously, e.g., mastitis and a respiratory infection.

The animals to be treated are, in particular, mammals such as cattle or pigs. The liquid preparation is preferably given as an intramuscular injectable solution.

The advantages associated with the present invention are manifold.

The invention for the first time provides a mixture of meloxicam and penethamate hydroiodide. The treatment of mammals with this mixture has not hitherto been possible as no stable formulations have been successfully prepared.

The preparation according to the invention is sufficiently stable, which is surprising as a 2% meloxicam solution, for example, has a pH of 8.9 and yet is highly stable in use.

This combined preparation in the form of the liquid preparation according to the invention enables two active substances to be given simultaneously, thereby allowing two indications such as mastitis and respiratory tract infections to be treated simultaneously. The application of the two active substances means that the amount of antibiotic can be reduced, compared with the amount of antibiotic which would have to be given separately, indicating a synergistic effect. One advantage of reducing the amount of antibiotic required is, for example, a significantly reduced antibiotic load on the farm animals treated, with a resulting significant reduction in the usual risks such as the development of allergies and resistance.

Moreover, the preparation can easily be produced using existing commercial preparations. One component of Ingel-Mamyzin®, i.e., penethamate hydroiodide in the form of a dry substance, can be used, and a standard commercial meloxicam solution may be used.

Moreover, a simple method of production is provided in which the liquid preparation according to the invention is produced immediately by combining and shaking the ingredients. Administration of the preparation in the form of an injectable solution confers advantages on the count of the accuracy of metering, the immediate bioavailability of the active substances, and the ease of administration, particularly when treating animals.

The Examples that follow serve to illustrate some formulations according to the invention. They are to be regarded as possible methods described by way of example without restricting the invention to their content.

### EXAMPLE 1

In order to investigate stability, microscope images were taken of one embodiment of the liquid preparation according to the invention and a formulation known from the prior art.

These are shown in FIGS. 1 to 5. Specifically:

FIG. 1 shows a microscope image of an Ingel-Mamyzin® suspension;

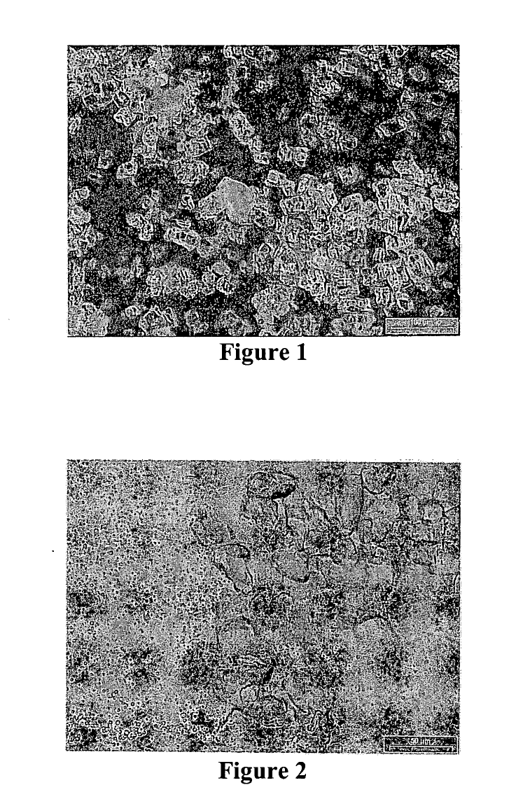

FIG. 2 shows a microscope image of an embodiment of the preparation according to the invention, immediately after manufacture;

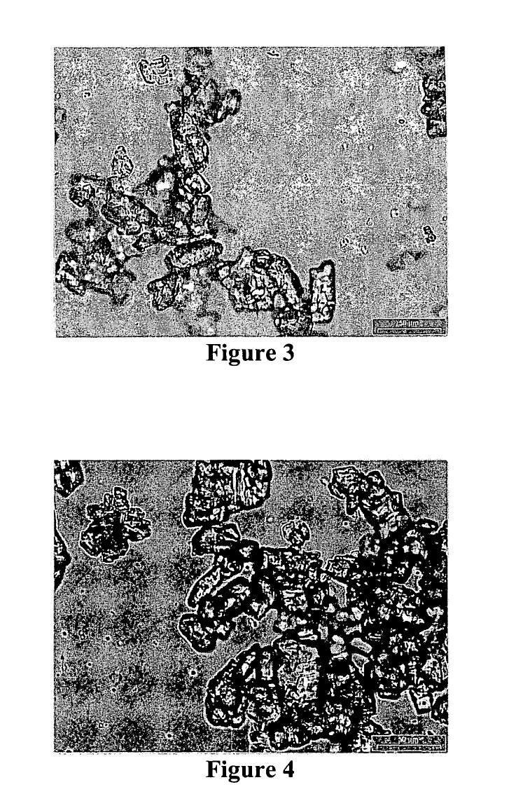

FIG. 3 shows a microscope image of an embodiment of the preparation according to the invention 6 hours after manufacture;

FIG. 4 shows a microscope image of an embodiment of the preparation according to the invention 24 hours after manufacture; and

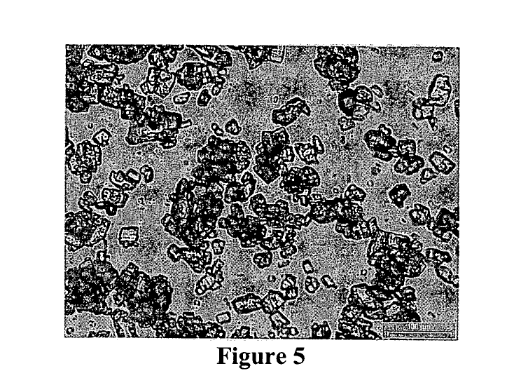

FIG. 5 shows a microscope image of an embodiment of the preparation according to the invention 4 days after manufacture followed by storage in the refrigerator at 4° C. to 8° C.

The microscope image in FIG. 1 shows an Ingel-Mamyzin® suspension produced immediately before the image was taken. The two vials supplied by the manufacturer, one vial containing methyl-4-hydroxybenzoate with water as solvent, and the other vial containing the dry substance penethamate hydroiodide, were combined and shaken. The resulting suspension was drawn up in a syringe and one drop of liquid was applied to the slide of a microscope. The image shows an intact system without any detectable decomposition of the suspended penethamate hydroiodide.

FIGS. 2 to 5 show an embodiment of the liquid preparation of the invention depending on the storage time. The preparation was produced from 15.6 mL of a 2% meloxicam solution to which 5 g of penethamate hydroiodide have been added as dry substance. The mixture was then shaken until a homogeneous mixture was obtained in the form of a suspension. The resulting suspension was drawn up with a syringe and one drop of liquid was applied to the slide of a microscope. The samples were taken immediately after production, after 6 hours, 24 hours, and 4 days after manufacture, stored in a refrigerator at a temperature of 4° C. to 8° C. All the images show an intact system without any detectable breakdown of the components. The suspension obtained hardly changed at all as a function of the storage time; the suspension was therefore still stable after 4 days and could thus be used without restriction.

### EXAMPLE 2

The intention was to examine the influence of the alkaline pH and the solvent on the stability of the penethamate. For this, a suspension according to the invention, prepared from meloxicam/penethamate hydroiodide in ethanol/water (sample solution), and a comparative solution obtained by the usual method of preparation from the two components of Ingel-Mamyzin®: solvent+dry substance were investigated using a specially developed HPLC method and in each case the content of penethamate, meloxicam, and Impurity II was determined. Impurity II is a breakdown product of meloxicam, i.e., 2-amino-5-methylthiazole, the increasing content of which indicates increased breakdown.

(a) HPLC Method

The following HPLC method was used:
Column: YMC ODS AQ, 120 A, 3 μm, 150×4 mm, without preliminary column
Flow: 0.8 mL/min
Temperature: 40° C.
Detection: 260 nm
DAD: Control peak purity, recording from 190-600 nm
Injection vol.: 10 μL
Mobile Phase: acetonitrile/buffer (33/67, V/V)
Buffer: 2.7 g (0.02 mol) of KH₂PO₄ and 4.4 g (0.02 mol) of 1-heptanesulfonic acid-sodium salt monohydrate are dissolved in about 800 mL of twice-distilled water (HPLC-grade). The solution is adjusted to pH=3 with conk phosphoric acid and then made up to 1000 mL.
   (b) Preparation of the Comparative Solution
(Ingel-Mamyzin®: solvent+dry substance)

The comparative solution was prepared by the following method:

Preparation of the Stock Solution:

1 vial of Ingel-Mamyzin® dry substance (5 g of penethamate hydroiodide) is combined with 1 vial of Ingel-Mamyzin® solvent (15.6 mL of water containing as preservative 23.4 mg of methyl-4-hydroxybenzoate per vial) and dissolved as thoroughly as possible with vigorous shaking (about 2 minutes). This suspension was quantitatively transferred into a 100 mL measuring flask with acetonitrile, 20 mL of placebo solution (2% meloxicam) was added, and the liquid was topped up to 100.0 mL with acetonitrile.

Dilution was carried out as follows:

1.0 mL of the freshly prepared stock solution was diluted with eluant (acetonitrile/buffer 33/67, v/v) to obtain 50.0 mL. The following concentrations were obtained:

c(Penethamate)=1.0 mg/mL

c(methyl-4-hydroxybenzoate)=0.00468 mg/mL

(c) Preparation of the Sample Solution

Liquid preparation according to the invention (Ingel-Mamyzin®-Dry substance+Meloxicam+Solvent)

The sample solution was prepared as follows:

Preparation of the Stock Solution

1 vial of Ingel-Mamyzin® dry substance (5 g of penethamate hydroiodide) was combined with 15.6 mL of a 2% meloxicam solution and dissolved as early as possible with vigorous shaking (about 2 minutes). This suspension was quantitatively transferred into a 100 mL measuring flask with acetonitrile, 20 mL of placebo solution (2% meloxicam) was added, and the liquid was topped up to 100.0 mL with acetonitrile.

Dilution was carried out as follows:

1.0 mL of the freshly prepared stock solution was diluted with eluant (acetonitrile/buffer 33/67, v/v) to obtain 50.0 mL. The following concentrations were obtained:

c(penethamate)=1.0 mg/mL

c(meloxicam)=0.0624 mg/mL

(d) Investigation of Stability

The stability in use, i.e., the stability of the suspension immediately after manufacture, was investigated as a double measurement on a sample and comparative solution under two sets of storage conditions (refrigerator and ambient temperature) after a given time window.

The HPLC tests to determine the content of penethamate, meloxicam and Impurity II were carried out after 0 (=start), 2, and 7 days by comparison with an external calibrating solution.

According to the design of the test, 1 mL of the sample and comparative solutions were taken on day 1, 2, and 5 and discarded. In the evaluation, the sample taken was added to the concentration found. The test design used is shown in Table 1:

TABLE 1
Test Design Used

| Parameter | Start | 1 day | 2 days | 5 days | 7 days |
|---|---|---|---|---|---|
| 2° C. to 8° C. | x | take 1 mL | take 1 mL | take 1 mL | x |
| | | sample of each | sample of each | sample of each | |
| | | and discard | and discard | and discard | |
| Prepare 5 | Prepare 2 stock | 15.6 mL − 1 | 14.6 mL − 1 | 13.6 mL − 1 | Prepare 2 stock |
| comparison | solutions and 2 | mL = 14.6 mL | mL = 13.6 mL | mL = 12.6 mL | solutions and 2 |
| solutions | test solutions and | | | | test solutions and |
| (TS¹⁾ + solvent) | chromatograph | | | | chromatograph |
| and 5 test | | | | | (1 vial is kept in |
| solutions | | | | | reserve) |
| (TS + 15.6 mL | | | | | |
| 2% meloxicam | | | | | |
| solution) | | | | | |
| 15° C. to 25° C. | — | take 1 mL | x | test concluded | test concluded |
| | | sample of each | | | |
| | | and discard | | | |
| Prepare 3 | — | 15.6 mL − 1 | Prepare 2 stock | | |
| comparison | | mL = 14.6 mL | solutions and 2 | | |
| solutions | | | test solutions and | | |
| (TS + solvent) | | | chromatograph | | |
| and 3 test | | | (1 vial is kept in | | |
| solutions | | | reserve) | | |
| (TS + 15.6 mL of | | | | | |
| 2% Metacam | | | | | |
| solution) | | | | | |
| Comments | Analysis of the | — | Analysis of the | — | Analysis of the |
| | comparative and | | comparative and | | comparative and |
| | test solutions | | test solutions | | test solutions |

¹⁾Dry substance

(e) Preparation of the Calibrating Solution

The results were evaluated against the following calibrating solution (n=2).

Preparation of the Stock Solution of Impurity II

About 2.4 mg of Impurity II were dissolved in 20.0 mL of twice-distilled water. The concentration was c=0.12 mg/mL.

About 100 mg of penethamate and about 6 mg of meloxicam were weighed into a 10 mL measuring flask, 2 mL of acetonitrile and 2 mL of placebo solution (2% meloxicam) and 0.5 mL of Impurity II stock solution were added (optionally treated in an ultrasound bath), and the mixture was made up to 10.0 mL with acetonitrile.

1.0 mL of this solution is diluted with eluant (acetonitrile/buffer 33/67, v/v) to 10.0 mL. The concentrations obtained were as follows:

c(penethamate)=1.0 mg/mL

c(meloxicam)=0.06 mg/mL

c(impurity II)=0.0006 mg/mL

(f) Summary of the Results

Table 2 below gives a summary of the results (average values of a double measurement with double injection) of the 3 analytes at investigation times t=0, 2, and 7 days.

TABLE 2
Summary of the Results

| | | Penethamate | | | | Impurity II | |
|---|---|---|---|---|---|---|---|
| | | Comp. | | Meloxicam | | Comp. | |
| | | sol'n. | Test sol'n. | Comp. | Test sol'n. | sol'n. | Test sol'n. |
| t | | [%] | [%] | sol'n. [%] | [%] | c[mg/mL] | c[mg/mL] |
| 0 | RT¹⁾ | 97.00 | 98.04 | — | 97.81 | — | 0.0001175 |
| 2 | RT¹⁾ | 96.58 | 91.64 | — | 98.69 | — | 0.0001318 |
| 7 | FR²⁾ | 99.78 | 99.47 | — | 101.92 | — | 0.0001129 |

¹⁾room temperature
²⁾refrigerator

The tests on the samples stored at 2° C.-8° C. showed no reduction in the content of penthamate and meloxicam after a storage period of 7 days.

When the preparations were stored at ambient temperature the content of penethamate decreased by about 6.4% compared with the initial test (t=0). The other two analytes meloxicam and Impurity II proved to be stable.

In order to monitor the peak purity, DAD spectra were recorded at a wavelength range of 190 to 600 nm and the match factor was determined. Peak purity is ensured with a match factor of >900. All the match factors were above 900.

1. A liquid composition comprising meloxicam, penethamate hydroiodide, and at least one solvent.

2. The liquid composition according to claim 1, wherein the meloxicam is dissolved in the solvent.

3. The liquid composition according to claim 1, wherein the solvent is alcohol, water, or an alcohol/water mixture.

4. The liquid composition according to claim 2, wherein the solvent is alcohol, water, or an alcohol/water mixture.

5. The liquid composition according to claim 3, wherein the alcohol is ethanol.

6. The liquid composition according to claim 4, wherein the alcohol is ethanol.

7. The liquid composition according to claim 1, wherein the solvent is a mixture of ethanol and water with a ratio of ethanol to water of 1:7 to 1:5.7.

8. The liquid composition according to claim 2, wherein the solvent is a mixture of ethanol and water with a ratio of ethanol to water of 1:7 to 1:5.7.

9. The liquid composition according to one of claims 1 to 8, wherein the liquid composition contains 0.2 g to 0.5 g of penethamate hydroiodide per mL of the liquid composition.

10. The liquid composition according to one of claims 1 to 8, wherein the liquid composition contains 0.2 g to 0.4 g of meloxicam per mL of the liquid composition.
